(19) United States
(12) Patent Application Publication
(10) Pub. No.: US 2013/0103010 A1
(43) Pub. Date: Apr. 25, 2013

(54) System and Method for Laser Ablation on a Surgical Surface

(71) Applicants: Robert Edward Grant, Laguna Beach, CA (US); David Haydn Mordaunt, Los Gatos, CA (US); Kristian Hohla, Muenchen (DE); Gwillem Mosedale, Muenchen (DE)

(72) Inventors: Robert Edward Grant, Laguna Beach, CA (US); David Haydn Mordaunt, Los Gatos, CA (US); Kristian Hohla, Muenchen (DE); Gwillem Mosedale, Muenchen (DE)

(21) Appl. No.: 13/405,069

(22) Filed: Feb. 24, 2012

Related U.S. Application Data
(60) Provisional application No. 61/549,706, filed on Oct. 20, 2011.

(51) Int. Cl. A61F 9/01; A61F 9/011; A61B 18/20

(52) U.S. Cl. 606/4; 606/17

(57) ABSTRACT

A system and method for performing Laser Induced Optical Breakdown (LIOB) on a target tissue includes a detector for imaging the interface surface between the target tissue and a base tissue. Also included is a laser unit for generating a laser beam, and for focusing the laser beam to a focal point. A computer is provided for controlling movement of the focal point. Specifically, this control is accomplished to maintain the focal point in the target tissue, but beyond a predetermined distance from the interface surface. For the present invention, the predetermined distance is established by considerations of laser beam geometry, which are applied in the context of images that are created of the interface surface.

### FIELD OF THE INVENTION

The present invention pertains generally to systems and methods for performing ophthalmic laser surgical procedures. More particularly, the present invention pertains to systems and methods that use visual imaging techniques to establish an interface surface between different types of eye tissue (i.e. a target tissue and a base tissue), and that use the resultant images as a reference for exclusively performing Laser Induced Optical Breakdown (LIOB) in only the target tissue. The present invention is particularly, but not exclusively, useful for laser surgical procedures wherein LIOB is performed in the target tissue beyond a predetermined distance form the tissue interface to avoid any LIOB of the base tissue.

### BACKGROUND OF THE INVENTION

During a surgical laser procedure, whenever Laser Induced Optical Breakdown (LIOB) is performed on ophthalmic tissue, control of the LIOB is crucial. For instance, it is just as important that there be no adverse effects on non-targeted tissue as it is that LIOB be properly and effectively performed on the intended target tissue. Stated differently, in an ophthalmic surgical procedure, it is important that LIOB be confined to only target tissue. This, however, may be difficult to accomplish for various reasons. For one, it often happens that two different types of tissue are juxtaposed and it is necessary to perform LIOB in only one of the tissues. Further, it may be desirable to perform this LIOB as close as possible to the interface between the two tissues.

The situation noted above for juxtaposed tissues is complicated by the fact that different types of ophthalmic tissue (e.g. vitreous and retina) typically have different LIOB thresholds. Moreover, it can happen that the upstream tissue (i.e. tissue through which the laser beam passes en route to the underlying target tissue) will have a lower LIOB threshold than the target tissue. In this case, the upstream tissue is clearly vulnerable to unintended LIOB.

In order to avoid the adverse effects of unintended LIOB, there are at least two interrelated factors that need to be considered. For one, it is essential that the interface surface between two different juxtaposed tissues be accurately identified. For another, it is also essential that the “fluence” (i.e. energy density) in the surgical laser beam never exceed the LIOB threshold of a non-target tissue as the laser beam passes through the non-target tissue. With this in mind, it is helpful to understand how the “fluence” of a laser beam will vary relative to the location of the laser beam's focal point. Specifically, this variation results because there is an inverse relationship between “fluence” and the cross section area of the laser beam. Thus, “fluence” increases in a downstream direction from the source of the laser beam as the cross section of the beam decreases toward the focal point. Continuing downstream from the focal point, however, the “fluence” will decrease as the cross section of the beam increases. Therefore, LIOB thresholds are properly considered both upstream and downstream from the laser beams' focal point.

In light of the above, it is an object of the present invention to provide a system and method for performing LIOB in one type of tissue beyond a distance “d” from an interface surface that is located between two different types of tissue. Still another object of the present invention is to control the location of a laser beam's focal point based on considerations of “fluence” in the laser beam. Yet another object of the present invention is to control the location of a laser beam's focal point using an entire surface image as a reference. Another object of the present invention is to provide a system and method for ablating a target tissue that is easy to implement, is simple to use and is comparatively cost effective.

### SUMMARY OF THE INVENTION

In accordance with the present invention, a system and method are provided for moving the focal point of a laser beam through eye tissue. Specifically, this is done for the purpose of performing Laser Induced Optical Breakdown (LIOB) on the tissue during a surgical procedure. As envisioned for the present invention, the path of the focal point will be through tissue that is inside the eye. Consequently, the target tissue for LIOB will be in a layer of underlying tissue, and the laser beam must necessarily pass through a layer of overlying tissue before it gets to the underlying target tissue. It typically happens that the overlying tissue and the underlying tissue will have different thresholds for LIOB. It is, nevertheless, desirable, and perhaps essential, that LIOB occur in only the target (i.e. underlying) tissue.

With this in mind, an operational concern for the present invention is that LIOB may be inadvertently performed on the overlying tissue. This is particularly problematic when the LIOB threshold of the overlying tissue is below the LIOB threshold of the underlying (target) tissue. In such a case, if the focal point of the laser beam is too close to the interface surface that is between the overlying tissue and the underlying (target) tissue, it can happen that the energy density (fluence) of the laser beam will exceed the LIOB threshold of the overlying tissue. As indicated above, this is to be avoided.

Structurally, a system in accordance with the present invention includes a laser unit for generating a laser beam with ultrashort pulses (e.g. femtosecond, picosecond or short nanosecond). Also, it includes an optical assembly for focusing the laser beam along a beam path. For example, the optical assembly may include scanners, adaptive optics, or optics with a variable numerical aperture. Importantly, this laser beam will have determinable cross sectional dimensions at respective stations along the beam path. Stated differently, depending on the energy in the laser beam, and the assembly of adaptive optics that is being used for the system, the laser beam will be dimensioned to have a determinable profile. Further, based on this profile, the energy density (i.e. fluence) of the laser beam at selected stations along the beam path can be determined.

In addition to the laser unit, the system also includes a detector for identifying a reference base inside an eye of a patient. For purposes of the present invention, this reference base is preferably an interface surface that is identified inside an eye, and is located between an overlying tissue and an underlying tissue. As noted above, the overlying tissue will have an LIOB threshold, “T₁”, and the underlying tissue will have a different LIOB threshold, “T₂”. Preferably, the detector will be an optical device that identifies the reference base (interface surface) using any of various well known imaging techniques. More specifically, imaging techniques envisioned for the present invention include optical, interferometric and ultrasound techniques. Further, these techniques may be employed by appropriately using Optical Coherence Tomography (OCT), confocal microscopy, Scheimpflug, two-photon imaging, or laser (optical) range finding devices.

A computer is also included in the system of the present invention and it will be used for controlling an operation of the laser unit in accordance with a predetermined computer program. Thus, the computer controls the movement of the laser beam's focal point. In particular, these movements may be in geometric and/or non-geometric patterns that include spirals, lines, rasters, circles, planes and cylinders.

The computer is also used to select a station on the beam path having a specified energy density (fluence). As envisioned for the present invention, the identification of a station involves its location on the beam path, as well as the cross sectional area of the laser beam at that location. Thus, for a beam having a particular energy, the energy density (fluence) of the beam at a particular station can be determined. This selection of a station for the present invention is important for at least two reasons. For one, the selected station will have an energy density (fluence) that is below the “T₁” LIOB threshold for the overlying tissue. For another, the selected station can be determined as being at a distance “d” upstream from the focal point of the laser beam.

An exemplary application of the present invention involves the cornea of an eye. In this example, the overlying tissue is the epithelium of an eye and the underlying surface is the stroma of the eye. Accordingly, the interface surface is against a posterior surface of the epithelium (Bowman's membrane). In one mode of operation, the computer maintains the distance “d” at a constant value in order to create a flap of stromal tissue having a substantially uniform thickness. Such a flap could be used, for example, as part of a LASIK procedure. In certain circumstances, a constant stromal thickness for such flaps, or even a predetermined stromal thickness pattern for such a flap, may be desirable in its own right. Thus, “d” can be established, according to the requirements of a particular application, to create patterns for stromal tissue that have predetermined thicknesses. For example, such applications may include LASIK procedures (as noted above), the creation of stromal pockets, and the creation of constant thickness flaps. In other modes of operation, the distance “d” can either be continuously minimized, or otherwise arbitrarily established. Within the eye, it will be appreciated that the interface surface may also be established between tissues in the lens of an eye, between tissues in the retina of an eye, or between tissues in the sclera.

In other aspects of the present invention, a femtosecond laser is operated to ablate target tissue to within an ultra-short distance from an interface between the target tissue and the surface of a base tissue. To do this, an imaging unit is employed for a two-fold purpose. For one, the imaging unit is used to create a three dimensional image of the interface surface of the base tissue. This image necessarily includes dimensional and location information about the interface surface. For another, the imaging unit is used to subsequently monitor movements of the laser beam focal point. Specifically, this monitoring is accomplished in real time, as the target tissue is being ablated. In particular, using the three dimensional image of the interface surface as an input reference, closed loop feedback control techniques are employed to maintain the laser beam's focal point in the target tissue. This is done to maintain the focal point beyond a predetermined distance “d” from the interface surface with the base tissue. Preferably, ablation of the target tissue is accomplished by Laser Induced Optical Breakdown (LIOB), and the imaging unit is an Optical Coherence Tomography (OCT) device.

One example of an ophthalmic application for the ablation of a target tissue at its interface with a base tissue involves the localized severance of vitreous bands (fibers) to prevent the creation of macular holes. In this case, it is known that with age, the vitreous shrinks inside the eye. When this happens, fibers (bands) within the vitreous can pull on the retina with the potential to tear the retina. The result of such a tear is the creation of macular holes that can eventually lead to a loss of sight. As envisioned for the present invention, a femtosecond laser can be used to sever localized vitreous bands next to the surface of the retina to prevent this from happening. For another example of an ophthalmic application, the femtosecond laser can be used to remove the remnants of crystalline lens that are left in the capsular bag after the crystalline lens has been removed from the bag during a cataract surgery.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic of the components of a system in accordance with the present invention;

FIG. 2A is an illustration of a laser beam and its focal point positioned relative to an overlying tissue layer and an underlying tissue layer during an operation of the present invention;

FIG. 2B is an illustration of a laser beam and its focal point positioned in an overlying tissue layer relative to an underlying tissue layer during an operation of the present invention;

FIG. 3 is a cross section view of an eye showing a femtosecond laser beam being employed to sever localized vitreous bands for the purpose of preventing the creation of macular holes in the retina of the eye; and

FIG. 4 is a cross section view of an eye showing a femtosecond laser beam being employed to ablate crystalline lens tissue in the capsular bag after a removal of the crystalline lens from the bag.

### DESCRIPTION OF THE PREFERRED EMBODIMENTS

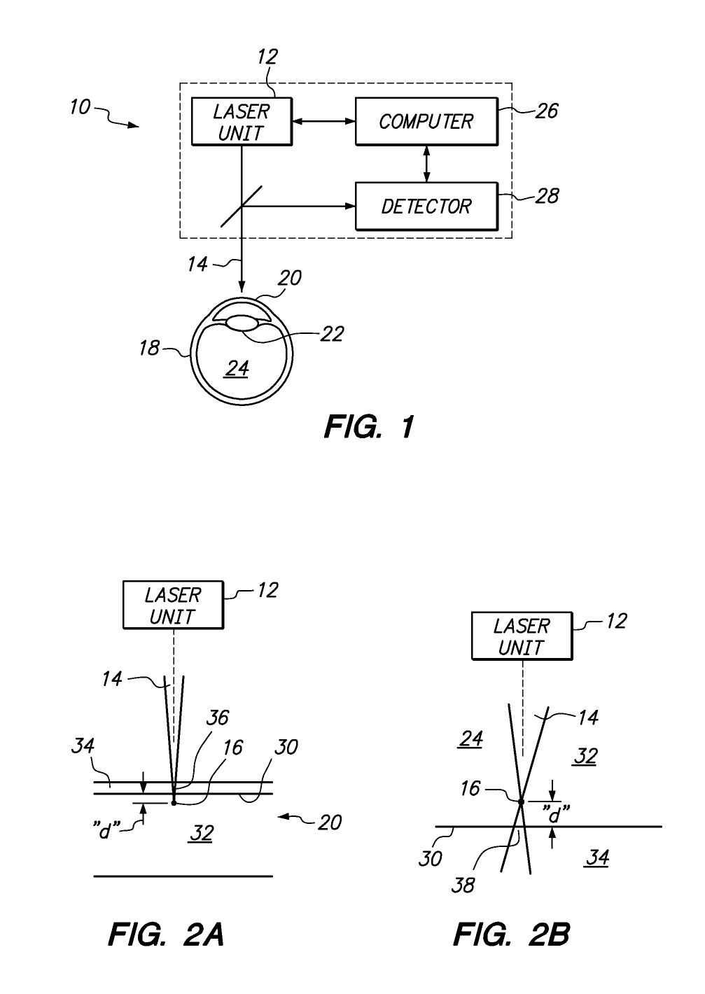

Referring initially to FIG. 1 a system in accordance with the present invention is schematically shown and is generally designated 10. As shown, the system 10 includes a laser unit 12 that is provided to generate a laser beam 14 which is directed and focused to a focal point 16 (e.g. see FIG. 2A). For the present invention, it is envisioned that the focal point 16 will be in a selected ophthalmic tissue of an eye 18. For example, the present invention envisions directing the focal point 16 into the cornea 20, the crystalline lens 22, or the vitreous 24 of eye 18 for performing different ophthalmic laser surgeries. Other locations and structures of the eye 18, though not specifically mentioned herein, are also contemplated. For the purposes of the present invention, the laser beam 14 is preferably a so-called femtosecond laser that is capable of being configured to perform Laser Induced Optical Breakdown (LIOB) on selected tissue in the eye 18.

FIG. 1 also shows that the system 10 includes a computer 26 and a detector 28. Insofar as the detector 28 is concerned, it is preferably an imaging device that employs Optical Coherence Tomography (OCT) techniques which allow the detector 28 to create three dimensional images of tissues in the eye 18. These images will preferably include dimensional and location information about the selected tissues of interest. More specifically and operationally important, the detector 28 needs to be capable of generating images that will distinguish between different types of tissues in the eye 18. In particular, the detector 28 must be capable of creating an image of an interface surface 30, such as the ones shown in FIG. 2A and FIG. 2B.

In FIG. 2A, a situation is presented wherein the focal point 16 of laser beam 14 is established in a target tissue 32 that is downstream from a base tissue 34. Such a situation would occur, for example, when the target tissue 32 is the stroma of cornea 20 and the base tissue 34 is the epithelium of the cornea 20. For instance, this type situation is confronted when it is desirable to create a superficial flap (i.e. a very thin flap) [not shown] on the anterior of the cornea 20, while removing as little stromal tissue as possible.

For any operation of the present invention, the interface surface 30 between the base tissue 34 and the target tissue 32 would be identified by the detector 28. Thereafter, movements of the focal point 16 within the target tissue 32 (i.e. stroma) are maintained by control from the computer 26. In line with the above discussions regarding a situation as depicted in FIG. 2A, movements of the focal point 16 in the target tissue 32 would be maintained beyond a distance “d” from the interface surface 30. Recall, in the portion of the laser beam 14 that is upstream from the focal point 16, the “fluence” of the laser beam 14 will increase in the direction toward the focal point 16. Thus, in this example, LIOB would be done in accordance with the present invention by maintaining “fluence” (i.e. energy density) at a cross section 36 of the laser beam 14 on the interface surface 30 that is below the LIOB threshold of the base tissue 34 (in this example, the base tissue 34 is the epithelium of cornea 20).

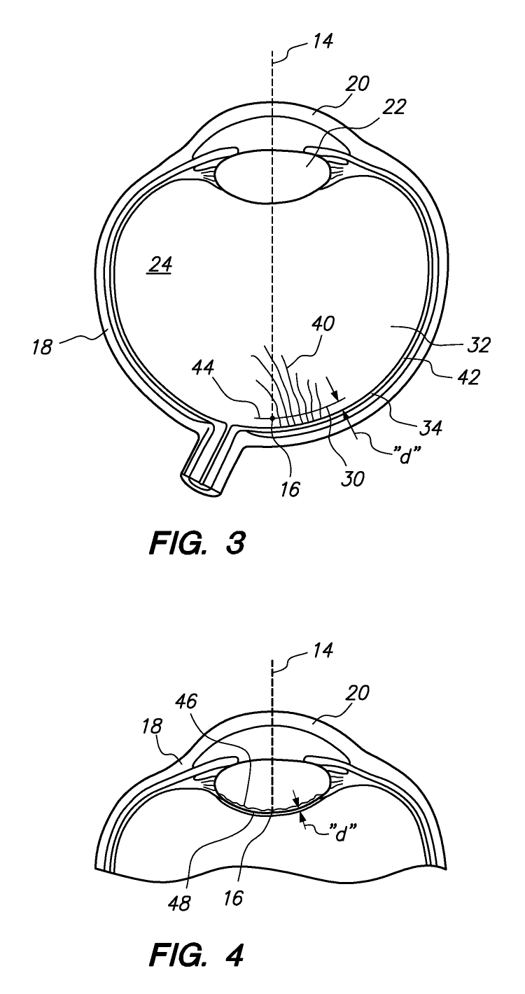

In FIG. 2B, the situation is effectively reversed when the target tissue 32 is upstream from the base tissue 34. Again, the interface surface 30 is identified between the target tissue 32 and the base tissue 34. In this case, however, the focal point 16 is located upstream from the interface surface 30. Thus, in order to avoid unintended LIOB of the base tissue 34, the “fluence” of laser beam 14 at a cross section 38 on the interface surface 30 must be below the LIOB threshold of the base tissue 34. Again, in line with the above discussions, movements of the focal point 16 in the target tissue 32 would be maintained beyond a distance “d” from the interface surface 30. Examples of operational applications, wherein the situation of FIG. 2B is depicted, are presented in FIG. 3 and FIG. 4.

In FIG. 3 the target tissue 32 is the vitreous 24 of eye 18 and, more particularly, vitreous fibers 40 within the vitreous 24. The base tissue 34 in this example is the retina 42. Further, in this example it is desirable to sever the fibers 40 from the retina 42 to prevent what is known in the pertinent art as “macular hole.” To do this, with the understanding that there can be no LIOB damage to the retina 42, a surgical surface 44 is established at the distance “d” from the interface surface 30. As shown in FIG. 2B, this is done by identifying the distance “d” downstream from the focal point 16 where the “fluence” of the laser beam 14 will be below the LIOB threshold of the base tissue 34 (i.e. the retina 42). Once this distance “d” has been identified, and is input to the computer 26 as a basis for the surgical surface 44, the focal point 16 of laser beam 14 can be moved over the surgical surface 44, through the target tissue 32 (i.e. vitreous 24), to cut the vitreous fibers 40, and thereby prevent “macular hole.”

In FIG. 4 the target tissue 32 includes remnants 46 of the crystalline lens 22 that may remain after a removal of the crystalline lens 22 in a cataract surgery. The base tissue 34 in this case is the posterior section of a capsular bag 48. Here, the surgical objective is to remove the remnants 46 from the capsular bag 48 prior to an implantation of an Intraocular Lens (IOL) [not shown]. In this case it, is desirable to preserve as much of the capsular bag 48 as possible for use in supporting the IOL. Again, the constraints considered above with reference to FIG. 2B are applicable. And control over the focal point 16 by the computer 26 is essentially the same as disclosed above.

While the particular System and Method for Laser Ablation on a Surgical Surface as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

1. A system for ablating a target tissue by Laser Induced Optical Breakdown (LIOB) which comprises:
a laser unit for generating a laser beam, wherein the laser unit includes a means for focusing the laser beam to a focal point and a means for selectively moving the focal point;
an imaging unit for creating a three dimensional image of an interface surface between a target tissue and a base tissue, wherein the image includes dimensional and location information of the interface surface;
a monitor connected with the imaging unit to follow the laser beam focal point, in real time, during an operation of the laser unit; and
a computer connected to the imaging unit and to the monitor for controlling the laser unit to move the focal point of the laser beam along a predetermined path in the target tissue, while maintaining the focal point beyond a predetermined distance “d” from the surface interface.

2. A system as recited in claim 1 wherein the base tissue is the retina of an eye and the target tissue is fibers in the vitreous of the eye.

3. A system as recited in claim 1 wherein the base tissue is the capsular bag of an eye and the target tissue is crystalline lens material.

4. A system as recited in claim 1 wherein the distance “d” is measured in an upstream direction from the interface surface toward the laser unit.

5. A system as recited in claim 1 wherein the distance “d” is measured to establish a fluence downstream from the focal point, at the interface surface, below an LIOB threshold for the base tissue.

6. A system as recited in claim 1 wherein the distance “d” is greater than ten microns.

7. A system as recited in claim 1 wherein the image includes dimensional and location information of the target tissue.

8. A system as recited in claim 1 wherein the monitor detects a deviation of the focal point from the predetermined path to create an error signal, and wherein the computer controls movement of the focal point to minimize the error signal.

9. A method for ablating target tissue in an eye of a patient which comprises the steps of:
imaging a volume of the target tissue, wherein the volume of target tissue has an outer surface;
identifying a three dimensional surgical surface during the imaging step, wherein the surgical surface has a predetermined area and is established within the volume of target tissue beyond a predetermined distance “d” from the outer surface of the volume of target tissue;
directing the focal point of a laser beam onto the surgical surface; and
moving the focal point of the laser beam along a predetermined path over the surgical surface to ablate target tissue at the surgical surface by Laser Induced Optical Breakdown (LIOB).

10. A method as recited in claim 9 wherein the volume of target tissue is the vitreous of the eye and the method further comprises the steps of:
locating vitreous bands in the vitreous of the eye during the imaging step, wherein the vitreous bands are connected to the retina of the eye; and
using the interface between the vitreous and the retina of the eye as the outer surface of the volume of target tissue.

11. A method as recited in claim 10 wherein the distance “d” is measured in an upstream direction from the outer surface.

12. A method as recited in claim 10 wherein the distance “d” is measured to establish fluence at the outer surface below the LIOB threshold for tissue of the retina.

13. A method as recited in claim 10 wherein the moving step is accomplished by moving the focal point of the laser beam along the predetermined path in the target tissue, and the method further comprises the steps of:
monitoring the focal point to detect deviations of the focal point from the predetermined path to create an error signal in response thereto; and
controlling the movement of the focal point to minimize the error signal.

14. A method as recited in claim 9 wherein the volume of target tissue includes remnants of the crystalline lens in the capsular bag after a lensectomy, and the method further comprises the step of continuing to move the focal point in conjunction with the moving step, to ablate tissue in the volume of target tissue.

15. A method as recited in claim 14 wherein the capsular bag is a base tissue and wherein the target tissue and the base tissue establish an interface surface therebetween.

16. A method as recited in claim 15 wherein the distance “d” is measured in an upstream direction from the interface surface.

17. A method as recited in claim 16 wherein the distance “d” is measured to establish a fluence at the interface surface below an LIOB threshold for tissue of the capsular bag.

18. A method as recited in claim 14 further comprising the steps of:
monitoring the focal point to detect deviations of the focal point from the predetermined path to create an error signal in response thereto; and
controlling the movement of the focal point to minimize the error signal.

19. A computer program product for ablating target tissue, wherein the computer program product comprises program sections for respectively: imaging a volume of the target tissue, wherein the volume of target tissue has an outer surface; identifying a three dimensional surgical surface, wherein the surgical surface has a predetermined area and is established within the volume of target tissue beyond a predetermined distance “d” from the outer surface of the volume of target tissue; directing the focal point of a laser beam onto the surgical surface; and moving the focal point of the laser beam along a predetermined path over the surgical surface to ablate target tissue at the surgical surface by Laser Induced Optical Breakdown (LIOB).

20. A computer program product as recited in claim 19 further comprising program sections for: measuring the distance “d” in an upstream direction from the interface surface toward the laser unit; establishing a fluence at the interface surface below an LIOB threshold for the base tissue; monitoring the focal point to detect deviations of the focal point from the predetermined path to create an error signal in response thereto; and controlling the movement of the focal point to minimize the error signal.
